(19) United States
(12) Patent Application Publication
(10) Pub. No.: US 2024/0086733 A1
(43) Pub. Date: Mar. 14, 2024

(54) METHOD FOR SEARCHING FOR NOVOLAC PHENOL RESIN, INFORMATION PROCESSING DEVICE, AND NON-TRANSITORY COMPUTER-READABLE RECORDING MEDIUM

(71) Applicant: DIC Corporation, Tokyo (JP)

(72) Inventor: Tomoyuki Imada, Chiba (JP)

(73) Assignee: DIC Corporation, Tokyo (JP)

(21) Appl. No.: 18/271,212

(22) PCT Filed: Dec. 8, 2022
(86) PCT No.: PCT/JP2022/045372

§ 371 Date: Jul. 6, 2023

(30) Foreign Application Priority Data
Dec. 17, 2021 (JP) 2021-205590

(51) Int. Cl. G06N 5/022; C08G 8/10

(52) U.S. Cl. CPC G06N 5/022; C08G 8/10

(57) ABSTRACT

Techniques for searching for phenol compounds are to be improved. A method for searching for a novolac phenol resin that is performed by an information processing device includes the steps of: generating a plurality of prediction models corresponding to a plurality of objective variables, using actual data pertaining to a novolac phenol resin; and searching for a novolac phenol resin having a desired physical property balance by inverse analysis using the prediction models. The actual data includes a polymer composition, a structural formula, a reaction solvent, and a reaction parameter pertaining to the novolac phenol resin. The objective variables include developability, heat resistance, and molecular weight.

### TECHNICAL FIELD

The present disclosure relates to a method for searching for a novolac phenol resin, an information processing device, and a non-transitory computer-readable recording medium. This application claims the priority based on Japanese Patent Application No. 2021-205590 filed in Japan on Dec. 17, 2021, the contents of which are incorporated herein by reference.

### BACKGROUND ART

Conventionally, resist materials made of resins with high insulation and heat resistance, such as polyimides, have been used for fine redistribution lines. One of technologies that meet the commercial demand for miniaturization is a technology using novolac phenol resins such as cresol novolac resins as additives. The novolac phenol resins for this application are required to have heat resistance (Tg), developability (ADR), and the like. However, these properties of novolac phenol resins are conflicting. It is therefore difficult to search for a novolac phenol resin that has a good balance of the required properties.

On the other hand, a technique for searching for a structure of a novel material based on a target physical property using a learned model is known, in which a material model is modeled and the material model is learned by machine learning (e.g., PTL 1).

### CITATION LIST

### Patent Literature

PTL 1: Japanese Patent No. 6832678

### SUMMARY OF INVENTION

### Technical Problem

The material search technique described in PTL 1 is targeted for general material models, and conventional technologies have not specifically focused on the search for novolac phenol resins such as cresol novolac resins used for semiconductor manufacturing.

An object of the present disclosure made in view of such circumstances is to improve the technology for searching for novolac phenol resins.

### Solution to Problem

A substance search method according to an embodiment of the present disclosure is a method for searching for a novolac phenol resin that is performed by an information processing device. The method includes the steps of:
generating a plurality of prediction models corresponding to a plurality of objective variables, using actual data pertaining to a novolac phenol resin; and
searching for a novolac phenol resin having a desired physical property balance by inverse analysis using the prediction models.

The actual data includes a polymer composition, a structural formula, a reaction solvent, and a reaction parameter pertaining to the novolac phenol resin.

The objective variables include developability, heat resistance, and molecular weight.

In the substance search method according to an embodiment of the present disclosure, in the step of generating the plurality of prediction models, a feature is calculated based on the actual data, and the feature is used as an explanatory variable for the prediction models.

In the substance search method according to an embodiment of the present disclosure, the feature includes at least one of a molecular fingerprint or a descriptor.

In the substance search method according to an embodiment of the present disclosure, the feature further includes information pertaining to an SP value of a solvent.

In the substance search method according to an embodiment of the present disclosure,
the actual data includes actual data of a novolac phenol resin used for a predetermined application and actual data of a novolac phenol resin used for other than the predetermined application, and
in the step of generating the plurality of prediction models,
after the prediction models are generated using the actual data of a novolac phenol resin used for other than the predetermined application, the prediction models are relearned using the actual data of a novolac phenol resin used for the predetermined application.

In the substance search method according to an embodiment of the present disclosure, the predetermined application is semiconductor manufacturing application.

An information processing device according to an embodiment of the present disclosure is an information processing device for searching for a novolac phenol resin. The information processing device includes a control unit.

The control unit
generates a plurality of prediction models corresponding to a plurality of objective variables, using actual data pertaining to a novolac phenol resin, and
searches for a novolac phenol resin having a desired physical property balance by inverse analysis using the prediction models.

The actual data includes a polymer composition, a reaction solvent, and a reaction parameter pertaining to the novolac phenol resin.

The objective variables include developability, heat resistance, and molecular weight.

A non-transitory computer-readable recording medium according to an embodiment of the present disclosure is a non-transitory computer-readable recording medium storing instructions.

The instructions, when executed by a processor, causes the processor to:
generate a plurality of prediction models corresponding to a plurality of objective variables, using actual data pertaining to a novolac phenol resin; and
search for a novolac phenol resin having a desired physical property balance by inverse analysis using the prediction models.

The actual data includes a polymer composition, a reaction solvent, and a reaction parameter pertaining to the novolac phenol resin.

The objective variables include developability, heat resistance, and molecular weight.

### Advantageous Effects of Invention

The method for searching for a novolac phenol resin, the information processing device, and the non-transitory computer-readable recording medium according to an embodiment of the present disclosure can improve the technology for searching for novolac phenol resins.

### BRIEF DESCRIPTION OF DRAWINGS

FIG. 1 is a diagram illustrating an overview of an embodiment of the present disclosure.

FIG. 2 is a block diagram illustrating an overall configuration of an information processing device that searches for a novolac phenol resin according to an embodiment of the present disclosure.

FIG. 3 is a flowchart illustrating the operation of a learning process of the information processing device that searches for a novolac phenol resin according to an embodiment of the present disclosure.

FIG. 4 is a flowchart illustrating the operation of a searching process of the information processing device that searches for a novolac phenol resin according to an embodiment of the present disclosure.

FIG. 5 is a GPC chart of a novolac phenol resin (A1) obtained in Synthesis Example 1.

FIG. 6 is a GPC chart of a novolac phenol resin (A2) obtained in Synthesis Example 2.

FIG. 7 is a GPC chart of a novolac phenol resin (A3) obtained in Synthesis Example 3.

FIG. 8 is a GPC chart of a novolac phenol resin (A4) obtained in Synthesis Example 4.

FIG. 9 is a GPC chart of a novolac phenol resin (A5) obtained in Synthesis Example 5.

FIG. 10 is a GPC chart of a novolac phenol resin (B1) obtained in Comparative Synthesis Example 1.

### DESCRIPTION OF EMBODIMENTS

A substance search method according to an embodiment of the present disclosure will be described below with reference to the drawings.

In the drawings, the same or corresponding parts are denoted by the same signs. In the description of the present embodiment, a description of the same or corresponding parts will be omitted or simplified as appropriate.

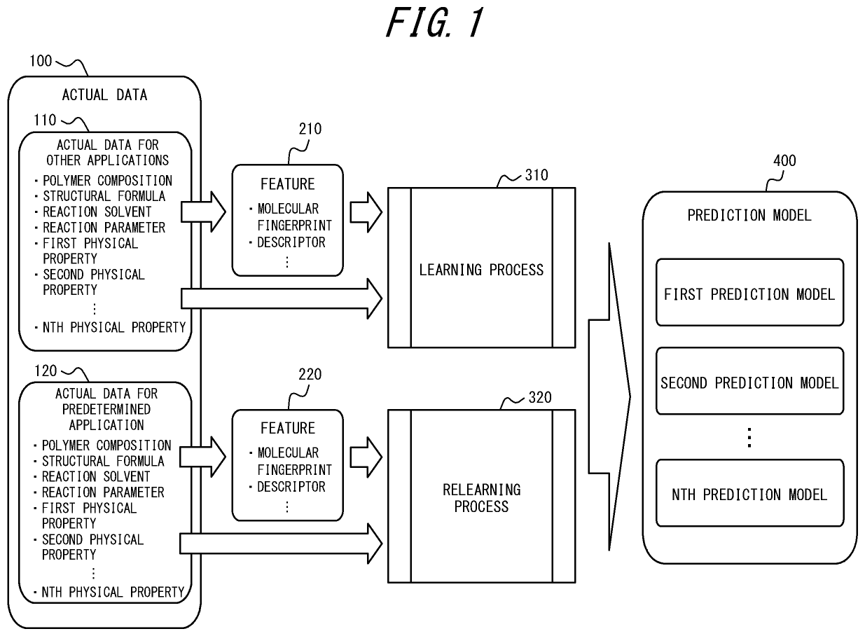

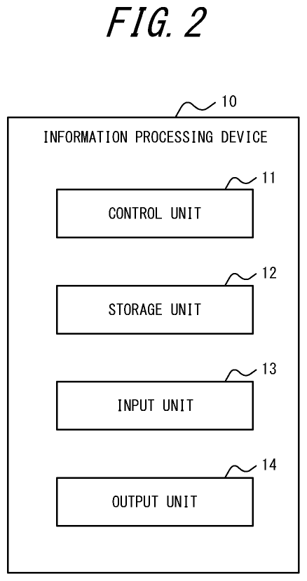

Referring to FIG. 1 and FIG. 2, an overview of a method for searching for a novolac phenol resin according to the present embodiment will be described.

First, an overview of the present embodiment will be described. In the substance search method according to the present embodiment, actual data 100 illustrated in FIG. 1 is used. The substance search method according to the present embodiment is performed by an information processing device 10 illustrated in FIG. 2. The information processing device 10 generates a plurality of prediction models 400 corresponding to a plurality of objective variables, using the actual data 100 pertaining to novolac phenol resins.

The actual data 100 includes actual data 120 for a predetermined application and actual data 110 for other applications. The actual data 120 for a predetermined application is, for example, actual data pertaining to novolac phenol resins for semiconductor manufacturing. That is, for example, the actual data 120 includes actual data pertaining to novolac phenol resins used for g- and i-line photoresists. The actual data 110 for other applications is actual data of novolac phenol resins used in applications other than the predetermined application (here, applications other than semiconductor manufacturing application). The actual data 120 for a predetermined application and the actual data 110 for other applications each include polymer compositions, structural formulas, reaction solvents, reaction parameters, and first to Nth physical properties pertaining to novolac phenol resins.

The first to Nth physical properties correspond to a plurality of objective variables. N is a positive integer. The information processing device 10 generates first to Nth prediction models corresponding to N objective variables. A plurality of objective variables include conflicting properties. For example, a plurality of objective variables include heat resistance (Tg), developability (ADR), and molecular weight.

An example of the heat resistance can be a glass transition temperature (Tg (° C.)). The developability can be an alkali dissolution rate (ADR (Å/s)) or a minimum exposure dose (J/cm²) at which a pattern with a predetermined length (e.g., 5 μm) is resolved. For example, when the developability is ADR, information in line with the evaluation of alkali developability described in Japanese Unexamined Patent Application No. 2021-152557 can be obtained. The molecular weight can be one or two or more selected from the group consisting of number average molecular weight (Mn), weight average molecular weight (Mw), peak top molecular weight (Mtop), and Z-average molecular weight (Mz).

First, the information processing device 10 performs a learning process 310 on a prediction model 400 using the actual data 110 for other applications. The information processing device 10 calculates a feature 210 from the actual data 110 for other applications. The information processing device 10 generates a plurality of prediction models 400 with the feature 210 as an explanatory variable and each physical property as an objective variable. Specifically, the information processing device 10 generates a first prediction model with the feature 210 as an explanatory variable and the first physical property as an objective variable. The information processing device 10 also generates a second prediction model with the feature 210 as an explanatory variable and the second physical property as an objective variable. In this way, the information processing device 10 generates the Nth prediction model with the feature 210 as an explanatory variable and the Nth physical property as an objective variable.

Next, the information processing device 10 performs a relearning process 320 on the prediction model 400 using the actual data 120 for a predetermined application. First, the information processing device 10 calculates a feature 220 from the actual data 120 for a predetermined application. The information processing device 10 performs the relearning process of each prediction model, using the feature 220 as an explanatory variable and each physical property as an objective variable. Specifically, the information processing device 10 performs the relearning process of the first prediction model, using the feature 220 as an explanatory variable and the first physical property as an objective variable. The information processing device 10 also performs the relearning process of the second prediction model, using the feature 220 as an explanatory variable and the second physical property as an objective variable. The information processing device 10 also performs the relearning process of the Nth prediction model, using the feature 220 as an explanatory variable and the Nth physical property as an objective variable. The information processing device 10 searches for a novolac phenol resin having a desired physical property balance by inverse analysis using the thus learned first to Nth prediction models.

In this way, according to the present embodiment, a plurality of prediction models are generated based on actual data pertaining to novolac phenol resins. Then, a novolac phenol resin having a desired physical property balance is searched for by inverse analysis using these prediction models. The search technology is thus improved in that a novolac phenol resin having a desired property balance can be searched for.

(Configuration of Information Processing Device)

The configuration of the information processing device 10 will now be described in detail. The information processing device 10 is any device used by a user. For example, a personal computer, a server computer, a general-purpose electronic device, or a dedicated electronic device can be employed as the information processing device 10.

As illustrated in FIG. 2, the information processing device 10 includes a control unit 11, a storage unit 12, an input unit 13, and an output unit 14.

The control unit 11 includes at least one processor, at least one dedicated circuit, or a combination thereof. The processor is a general-purpose processor such as a central processing unit (CPU) or a graphics processing unit (GPU), or a dedicated processor specialized for particular processing. The dedicated circuit is, for example, a field-programmable gate array (FPGA) or an application specific integrated circuit (ASIC). The control unit 11 performs a process related to the operation of the information processing device 10 while controlling each unit of the information processing device 10.

The storage unit 12 includes at least one semiconductor memory, at least one magnetic memory, at least one optical memory, or a combination of at least two of these. The semiconductor memory is, for example, a random access memory (RAM) or a read only memory (ROM). The RAM is, for example, a static random access memory (SRAM) or a dynamic random access memory (DRAM). The ROM is, for example, an electrically erasable programmable read only memory (EEPROM). The storage unit 12 functions, for example, as a main storage, an auxiliary storage, or a cache memory. The storage unit 12 stores data to be used in the operation of the information processing device 10 and data obtained by the operation of the information processing device 10.

The input unit 13 includes at least one input interface. The input interface is, for example, physical keys, capacitive keys, a pointing device, or a touch screen integrated with a display. The input interface may be, for example, a microphone that accepts a voice input or a camera that accepts a gesture input. The input unit 13 accepts an operation to input data to be used in the operation of the information processing device 10. The input unit 13 may be connected to the information processing device 10 as an external input device, rather than being included in the information processing device 10. For example, any method such as universal serial bus (USB), high-definition multimedia interface (HDMI) (registered trademark), or Bluetooth (registered trademark) can be used as the connection method.

The output unit 14 includes at least one output interface. The output interface is, for example, a display that outputs information in the form of images. The display is, for example, a liquid crystal display (LCD) or an electro luminescence (EL) display. The output unit 14 displays and outputs data obtained by the operation of the information processing device 10. The output unit 14 may be connected to the information processing device 10 as an external output device, rather than being included in the information processing device 10. For example, any method such as USB, HDMI (registered trademark), or Bluetooth (registered trademark) can be used as the connection method.

The functions of the information processing device 10 are implemented by executing a program according to the present embodiment on a processor corresponding to the information processing device 10. In other words, the functions of the information processing device 10 are implemented by software. The program causes a computer to perform the operation of the information processing device 10 to allow the computer to function as the information processing device 10. In other words, the computer functions as the information processing device 10 by performing the operation of the information processing device 10 under instructions of the program.

In the present embodiment, the program can be recorded on a computer-readable recording medium. The computer-readable recording medium includes a non-transitory computer-readable medium, for example, a magnetic recording device, an optical disk, a magneto-optical recording medium, or a semiconductor memory. The program is distributed, for example, by selling, transferring, or lending a portable recording medium such as a digital versatile disc (DVD) or a compact disc read only memory (CD-ROM) having the program recorded thereon. The program may be distributed by storing the program in a storage of an external server and transmitting the program from the external server to other computers. The program may be provided as a program product.

Some or all of the functions of the information processing device 10 may be implemented by a dedicated circuit corresponding to the control unit 11. In other words, some or all of the functions of the information processing device 10 may be implemented by hardware.

In the present embodiment, the storage unit 12 stores the actual data 100, the features 210 and 220, and the prediction models 400. The features 210 and 220 are calculated based on polymer compositions, structural formulas, reaction solvents, and reaction parameters of the actual data 100.

The feature 210 may include any data that represents the characteristics of a novolac phenol resin. For example, the feature 210 may include at least one of a molecular fingerprint or a descriptor. The feature 210 may include any data that represents the characteristics of a solvent.

For example, the feature 210 may include information pertaining to the SP value of a reaction solvent. The information pertaining to the SP value of a reaction solvent may include, for example, at least one of the SP value of a reaction solvent, the SP value of a final solvent, or the interaction term of the SP value.

The feature 220 may include any data that represent the characteristics of a novolac phenol resin. For example, the feature 220 may include at least one of a molecular fingerprint or a descriptor. The feature 220 may include any data that represents the characteristics of a solvent. For example, the feature 220 may include information pertaining to the SP value of a reaction solvent. The information pertaining to the SP value of a reaction solvent may include, for example, at least one of the SP value of a reaction solvent, the SP value of a final solvent, or the interaction term of the SP value.

The actual data 100, the features 210 and 220, and the prediction models 400 may be stored in an external device separate from the information processing device 10. In this case, the information processing device 10 may include an external communication interface. The communication interface may be an interface for either wired communication or wireless communication. In the case of wired communication, the communication interface is, for example, a LAN interface or a USB. In the case of wireless communication, the communication interface is, for example, an interface compatible with mobile communication standards such as LTE, 4G, or 5G, or an interface compatible with short-range wireless communication such as Bluetooth (registered trademark). The communication interface can receive data to be used in the operation of the information processing device 10 and can transmit data obtained by the operation of the information processing device 10.

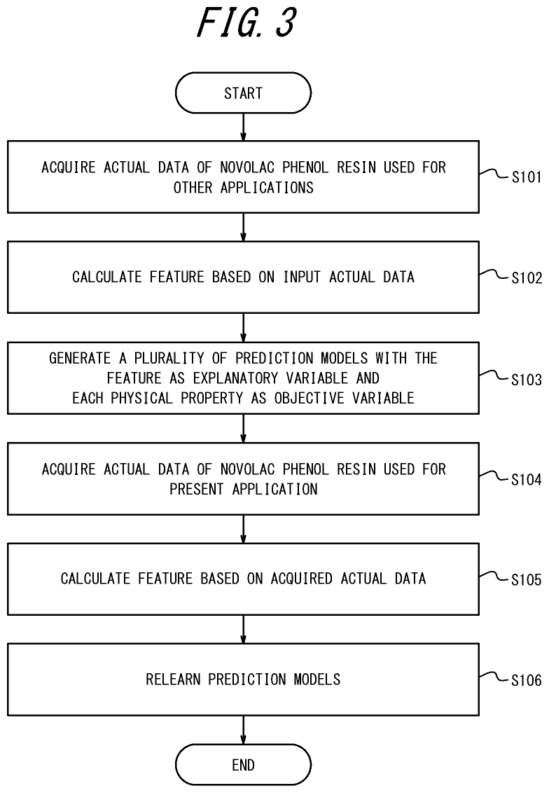

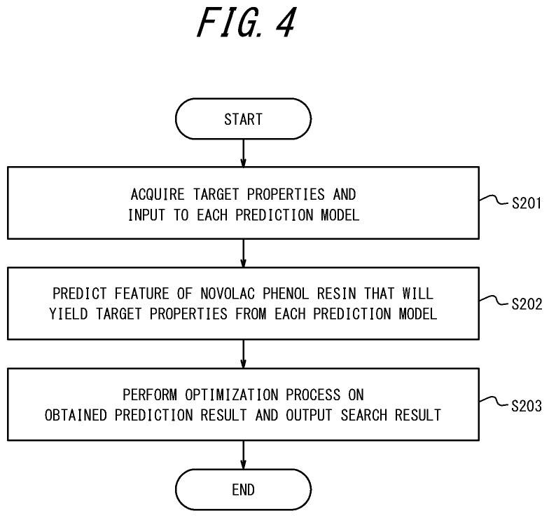

(Operation of Information Processing Device) Referring to FIG. 3 and FIG. 4, the operation of the information processing device 10 in the present embodiment will be described. FIG. 3 is a flowchart illustrating an example of the learning process and the relearning process performed by the information processing device 10 according to the present embodiment. FIG. 4 is a flowchart illustrating the search process performed by the information processing device 10 according to the present embodiment. Referring first to FIG. 3, an example of the learning process and the relearning process performed by the information processing device 10 will be described.

Step S101: The control unit 11 of the information processing device 10 acquires the actual data 110 of novolac phenol resins used in other applications. Any method can be employed to acquire the actual data 110. For example, the control unit 11 may acquire the actual data 110 by accepting input of actual data from the user through the input unit 13. For example, the control unit 11 may acquire the actual data 110 from an external device storing the actual data 110, via the communication interface.

Step S102: The control unit 11 calculates the feature 210 based on the input actual data 110. Specifically, the control unit 11 calculates the feature 210, based on the polymer compositions, structural formulas, reaction solvents, and reaction parameters included in the actual data 110. The control unit 11 may refer to a database or the like, if necessary, for calculation of the feature 210. In this case, such a database may be stored in the storage unit 12.

Step S103: The control unit 11 generates a plurality of prediction models 400 (first to Nth prediction models) with the calculated feature 210 as an explanatory variable and each physical property as an objective variable. Examples of the prediction models include, but not limited to, prediction models such as support vector machines, linear models, and nonlinear models. For example, the prediction model 400 may be a model generated based on a multilayer perceptron consisting of an input layer, a hidden layer, and an output layer. Alternatively, the prediction model 400 may be a model generated based on a machine learning algorithm such as convolutional neural network (CNN), recurrent neural network (RNN), or other deep learning.

Step S104: The control unit 11 acquires the actual data 120 of novolac phenol resins used in the present application. Any method can be employed to acquire the actual data 120. For example, the control unit 11 may acquire the actual data 120 by accepting input of actual data from the user through the input unit 13. For example, the control unit 11 may acquire the actual data 120 from an external device storing the actual data 120, via the communication interface. The amount of the actual data 120 may be smaller than the amount of the actual data 110.

Step S105: The control unit 11 calculates the feature 220 based on the input actual data 120. Specifically, the control unit 11 calculates the feature 220, based on the polymer compositions, structural formulas, reaction solvents, and reaction parameters included in the actual data 120. The control unit 11 may refer to a database or the like, if necessary, for calculation of the feature 210. In this case, such a database may be stored in the storage unit 12.

Step S106: The control unit 11 relearns a plurality of prediction models 400 (first to Nth prediction models) generated at step S103, using the calculated feature 220 as an explanatory variable and each physical property as an objective variable. In this way, the prediction models 400 according to the present embodiment are constructed. To distinguish between a plurality of prediction models 400 generated at step S103 and a plurality of prediction models 400 generated at step S104, they are also referred to as “generic prediction models” and “present prediction models”, respectively, in the present embodiment.

The prediction models 400 constructed by the above process may be verified for accuracy based on known data. As a result of the verification, if the accuracy is within a practical range, the process for searching for a novolac phenol resin using the prediction models 400 may be performed.

Referring now to FIG. 4, an example of the process for searching for a novolac phenol resin that is performed by the information processing device 10 will be described. In outline, the information processing device 10 searches for a novolac phenol resin having a desired physical property balance by inverse analysis using a plurality of prediction models 400.

Step S201: The control unit 11 of the information processing device 10 acquires physical properties pertaining to a desired novolac phenol resin (hereinafter referred to as target properties) and inputs the acquired physical properties to the prediction models 400 (first to Nth prediction models). For example, the control unit 11 acquires the target properties by accepting input of target properties from the user through the input unit 13.

Step S202: The control unit 11 uses the prediction models 400 to predict the feature of a novolac phenol resin that will yield the target properties acquired at step S201.

Step S203: The control unit 11 performs an optimization process on the prediction result obtained at step S202 and outputs the search result through the output unit 14. For example, the control unit 11 outputs the composition and synthesis method pertaining to a novolac phenol resin having a desired physical property balance, as the search result, through the output unit 14. Alternatively, the control unit 11 may output the feature pertaining to at least one novolac phenol resin having a desired physical property balance, as the search result, through the output unit 14.

Here, as the optimization process, the evaluation function can be maximized or minimized by gradient descent, Bayesian optimization, Gaussian process optimization, Python libraries such as GPyOpt, Optuna, and HyperOpt using their mechanisms, or genetic algorithms. However, the embodiment is not limited to these methods, and one or more methods that are appropriate for a target to be optimized can be selected.

In this way, according to the present embodiment, a plurality of prediction models 400 are generated based on the actual data 100 pertaining to novolac phenol resins. Then, a novolac phenol resin having a desired physical property balance can be searched for by inverse analysis using these prediction models 400. For example, a novolac phenol resin having desired heat resistance and desired developability can be easily searched for.

A novolac phenol resin may be searched for based on the experience and intuition of a person in charge, without using the search method according to the present embodiment. In this case, preliminary experiments for known novolac phenol resins and unknown novolac phenol resins are conducted, and least-squares regression calculation is performed on the experimental result to ascertain the correlation of given conditions and physical properties based on the experience and intuition of a person in charge. Then, after the correlation is ascertained, synthesis experiments of several novolac phenol resins pertaining to the search candidates are conducted. Least-squares regression calculation is further performed on the result of the experiments. These steps may be repeated to search for a desired novolac phenol resin. However, such a method is dependent on the experience and intuition of the person in charge and requires a large number of man-hours for the preliminary experiments and the experiments, and typically, it takes several months to conduct a single optimum composition search. On the other hand, according to the present embodiment, desired novolac phenol resins can be searched for in parallel and in a short time in the information processing device 10 based on the learned prediction models 400. As a result, the development man-hours can be significantly reduced.

In the present embodiment, the actual data 100 includes the actual data 110 for other applications and the actual data 120 for a predetermined application, and the learning process and the relearning process of the prediction models 400 are performed using these pieces of actual data. In this way, since a wider range of training data than in the present application is used in the learning process, it is possible to prevent decrease in accuracy due to extrapolation. In addition, the training data is limited to the present application in the relearning process. In this way, highly accurate prediction models can be generated in the search for a novolac phenol resin for a predetermined application. In the present embodiment, both the learning process and the relearning process are performed. However, as long as the accuracy is within a practical range, only the learning process is performed and the relearning process is not necessarily performed. In the learning process, at least one of the actual data for a predetermined application or the actual data for other applications may be used. In this way, the prediction models can be generated in a shorter time.

In the present embodiment, the feature may include at least one of a molecular fingerprint or a descriptor. Since the molecular fingerprint or the descriptor can indicate the characteristics of a novolac phenol resin, the accuracy of the prediction model 400 can be improved using the feature as an explanatory variable.

In the present embodiment, the feature may include information pertaining to the SP value of a reaction solvent. Since the information pertaining to the SP value of a reaction solvent can indicate the characteristics of the solvent in a synthesis reaction, the accuracy of the prediction model 400 can be improved using the feature as an explanatory variable.

In this description, the SP value ((J/cm³)^{1/2}) is expressed as the square root of (the cohesive energy density (that is, evaporation energy)) and may be calculated from a physical property value or calculated from a molecular structure. The SP value that can be used in the present embodiment is, for example, Hildebrand SP value (calculated by Hildebrand Rule), or calculated from a physical property value such as the value of latent heat of evaporation, surface tension, or solubility and the value of refractive index, or Hansen's HSP (calculated by Hansen's calculation method), or calculated by a method using a molecular structure, such as Small's calculation method, Rheineck and Lin's calculation method, Krevelen and Hoftyzer's calculation method, Fedors' calculation method, or Hoy's calculation method.

As the SP value of a solvent (encompassing a reaction solvent and a final solvent) in the present embodiment, one or a combination of two or more of the SP values calculated by the methods above can be used. When a solvent mixture of two or more solvents is used, the interaction term of the SP value is obtained, for example, by calculating the SP value of each solvent by the above method, then selecting one reference solvent serving as a reference, and calculating the difference between each of the other solvents and the reference solvent. Furthermore, when the interaction term of the SP value between a novolac phenol resin or its raw material component and a solvent is calculated, their respective SP values are calculated by the above method, then one reference substance serving as a reference is selected from the resin, the raw material component, or the solvent, and the difference between the reference substance and each component is calculated.

The novolac phenol resin in the present embodiment is a resin produced from the condensation of an aromatic compound having a phenolic hydroxyl group and a compound having an aldehyde group. The novolac phenol resin therefore has one or two or more kinds of a structural unit (A1) derived from an aromatic compound having a phenolic hydroxyl group and one or two or more kinds of a structural unit (A2) derived from an aldehyde group-containing compound. A preferred novolac phenol resin of the present embodiment contains a structural unit represented by the following general formula (1) as a main component.

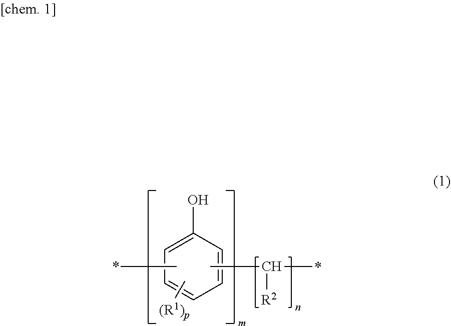

(In the above general formula (1), R¹ independently represents an amino group, a cyano group, or an alkyl group having 1 to 10 carbon atoms, where —CH₂— in the alkyl group having 1 to 10 carbon atoms is optionally substituted with —O—, —CO—, or —S— unless adjacent to each other, and R² independently represents a hydrogen atom, an alkyl group having 1 to 10 carbon atoms, or a phenyl group unsubstituted or optionally substituted with an alkyl group having 1 to 6 carbon atoms, where —CH₂— in the alkyl group having 1 to 6 carbon atoms is optionally substituted with —O—, —CO—, or —S— unless adjacent to each other,

p represents an integer of 0 or more and 3 or less, m represents the number of repeating units, preferably 5 to 150, and n represents the number of repeating units, preferably 5 to 150.)

In the above general formula (1), a plurality of R's may be the same or different from each other. Similarly, a plurality of Res may be the same or different from each other. The “main component” means that 51% by mass or more is contained with respect to the total (100% by mass) of the novolac phenol resin, preferably 73% by mass or more, more preferably 87% by mass, and even more preferably 93% by mass or more is contained.

In the novolac phenol resin having a structural unit represented by the above general formula (1), the composition ratio between the structural unit (A1) derived from an aromatic compound having a phenolic hydroxyl group (hereinafter also referred to as the structural unit (A1) having the number of repeating units m derived from an aromatic compound having a phenolic hydroxyl group) and the structural unit (A2) derived from an aldehyde group-containing compound (hereinafter also referred to as the structural unit (A2) having the number of repeating units n derived from an aldehyde group-containing compound) is preferably 80 to 150 parts by mass of the structural unit (A2) with respect to 100 parts by mass of the structural unit (A1).

A more preferred novolac phenol resin of the present embodiment contains a structural unit represented by the following general formula (2) as a main component.

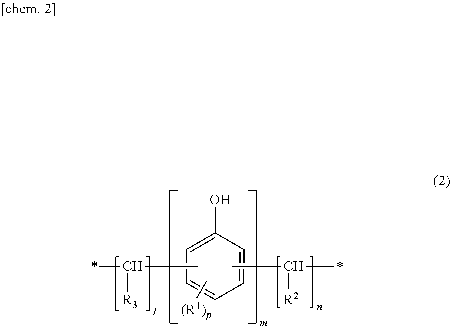

(In the above general formula (2), R³ independently represents a hydrogen atom, an alkyl group having 1 to 10 carbon atoms, or a phenyl group unsubstituted or optionally substituted with an alkyl group having 1 to 6 carbon atoms, where —CH₂— in the alkyl group having 1 to 6 carbon atoms is optionally substituted with —O—, —CO—, or —S— unless adjacent to each other, and l represents the number of repeating units, preferably 10 to 100. R¹, R², p, m, and n have the same meaning as those in the above general formula (1). R² and R³ are groups different from each other.)

In the above general formula (2), a plurality of R's may be the same or different from each other. Similarly, a plurality of Res may be the same or different from each other. Furthermore, a plurality of R³s may be the same or different from each other.

The novolac phenol resin having a structural unit represented by the above general formula (1) represents a copolymer having at least one kind of structural unit (A1) derived from an aromatic compound having a phenolic hydroxyl group and at least one kind of structural unit (A2) derived from an aldehyde group-containing compound. On the other hand, the novolac phenol resin having a structural unit represented by the above general formula (2) is an example of a preferred form of the novolac phenol resin having a structural unit represented by the above general formula (1), and represents a three- or more-component copolymer having one kind of structural unit (A1) derived from an aromatic compound having a phenolic hydroxyl group and two kinds of structural units (A2-1) and (A2-2) derived from an aldehyde group-containing compound.

In the novolac phenol resin having a structural unit represented by the above general formula (2), the composition ratio between the structural unit (A1) having the number of repeating units m derived from an aromatic compound having a phenolic hydroxyl group, the structural unit (A2-1) having the number of repeating units n derived from an aldehyde group-containing compound, and the structural unit (A2-2) having the number of repeating units l derived from an aldehyde group-containing compound is preferably 10 to 90 parts by mass of the structural unit (A2-1) with respect to 100 parts by mass of the structural unit (A1). It is preferable that 10 to 90 parts by mass of the structural unit (A2-2) is contained with respect to 100 parts by mass of the structural unit (A1). In this case, in total, 30 to 150 parts by mass of (A2-1) and (A2-2) is contained with respect to 100 parts by mass of (A1).

The novolac phenol resin in the present embodiment may be a random polymer, a block polymer, or an alternating polymer.

### EXAMPLES

(Learning Process and Relearning Process) A specific example of the learning process and the relearning process of novolac phenol resins according to the present embodiment will be described below. First, the actual data 110 for other applications and the actual data 120 for the present application pertaining to novolac phenol resins were stored in the storage unit 12. As described above, the actual data 110 for other applications and the actual data 120 for the present application include polymer compositions, structural formulas, reaction solvents, reaction parameters, and the first to Nth physical properties (objective variables) pertaining to novolac phenol resins.

The structural formulas pertaining to novolac phenol resins included in the actual data 110 for other applications and the actual data 120 for the present application according to the present example are the structures of the structural unit (A1) such as phenol, o-cresol, p-cresol, m-cresol 2,3-xylenol, 2,5-xylenol, 3,4-xylenol, 3,5-xylenol, 2,3,5-trimethylphenol, and 3,4,5-trimethylphenol, the structures of the structural unit (A2-1) containing no hydroxyl group, such as formalin, paraformaldehyde, acetaldehyde, chloroacetaldehyde, and benzaldehyde, which are aldehydes, and the structural formulas of the structural unit (A2-2) containing a hydroxyl group, such as salicylaldehyde, 4-hydroxybenzaldehyde, and 3-hydroxybenzaldehyde. The structural formulas are represented by SMILES strings. Based on the data, molecular fingerprints are calculated as features. ECFP2 fingerprints are used to calculate the molecular fingerprints. In this way, the structural units (A1), (A2-1), and (A2-2) are represented as a set of vectors. The respective parts by mass of the above structural units are stored in the storage unit 12 as the polymer composition according to the present example.

The reaction solvents included in the actual data 110 for other applications and the actual data 120 for the present application according to the present example are data of catalyst species. The reaction parameters included in the actual data 110 for other applications and the actual data 120 for the present application according to the present example are data describing the reaction process, such as reaction scale, temperature increase rate, reaction temperature, and catalyst removing step. Here, in the present example, the ratio of the SP value of the reaction solvent to the SP value of the final solvent (reaction solvent SP/final solvent SP) is included in the feature pertaining to the actual data 120 for the present application.

The first to Nth physical properties (objective variables) included in the actual data 110 for other applications according to the present example are the heat resistance (Tg as measured by DSC), ADR (A/sec), and weight average molecular weight (Mw) of a novolac phenol resin that was actually produced in other applications in the past.

The first to Nth physical properties (objective variables) included in the actual data 120 for the present application according to the present example are the heat resistance (Tg as measured by DSC), ADR (A/sec), and weight average molecular weight (Mw) of a novolac phenol resin that was actually produced in the present application in the past.

Next, a generic prediction model according to the present example was constructed using the actual data 110 for other applications described above. The coefficient of determination (R\hat{ }2 value) representing the consistency between prediction and actual measurement for the generic prediction model according to the present example was 0.60 to 0.70.

Subsequently, a present prediction model according to the present example was generated by relearning the generic prediction model according to the present example, using the actual data 120 for the present application. The coefficient of determination (R\hat{ }2 value) representing the consistency between prediction and actual measurement for the present prediction model according to the present example was 0.75 to 0.95.

(Search Process)

Examples 1 to 5 below are specific examples of using the present prediction model according to the present example to search for a result (recipe candidate) having target properties to be targeted. Comparative Example 1 is a specific example of using the generic prediction model according to the present example to search for a result (recipe candidate) having target properties to be targeted.

Here, Bayesian optimization was used as the optimization process, and grid search was performed on the neighborhood of a search candidate obtained by Bayesian optimization.

### Example 1

A candidate recipe was searched for by using the present prediction model according to the present example, selecting reaction materials: m-cresol, benzaldehyde, and salicylaldehyde, acidic catalyst: p-toluenesulfonic acid, reaction solvent: ethanol, and final solvent: γ-butyllactone, and setting heat resistance (Tg 150° C. or higher as measured by DSC), ADR 1000 Å/sec, and weight average molecular weight (Mw) 3000 as targets. A synthesis method based on the candidate recipe will be described in Synthesis Example 1.

### Example 2

A candidate recipe was searched for by performing the same operation except that the targets in Example 1 were changed to ADR 2300 Å/sec and weight average molecular weight (Mw) 2300. A synthesis method based on the candidate recipe will be described in Synthesis Example 2.

### Example 3

A candidate recipe was searched for by performing the same operation except that the targets in Example 1 were changed to ADR 900 Å/sec and weight average molecular weight (Mw) 2800. A synthesis method based on the candidate recipe will be described in Synthesis Example 3.

### Example 4

A candidate recipe was searched for by performing the same operation except that the targets in Example 1 were changed to ADR 6000 Å/sec and weight average molecular weight (Mw) 3000. A synthesis method based on the candidate recipe will be described in Synthesis Example 4.

### Example 5

A candidate recipe was searched for by performing the same operation except that the reaction solvent in Example 1 was changed to 250 g of ethanol, 30 g of 1-propanol, and 15 g of 2-propanol and the targets were changed to ADR 1000 Å/sec and weight average molecular weight (Mw) 3100. A synthesis method based on the candidate recipe will be described in Synthesis Example 5.

### Comparative Example 1

A candidate recipe was searched for by using the generic prediction model according to the present example, selecting m-cresol, benzaldehyde, salicylaldehyde, p-toluenesulfonic acid, and ethanol as a reaction solvent, and setting heat resistance (Tg 150° C. or higher as measured by DSC), ADR 1000 Å/sec, and weight average molecular weight (Mw) 3000 as targets. A synthesis method based on the candidate recipe will be described in Comparative Synthesis Example 1.

### Synthesis Example 1: Synthesis of Novolac Phenol Resin (A1)

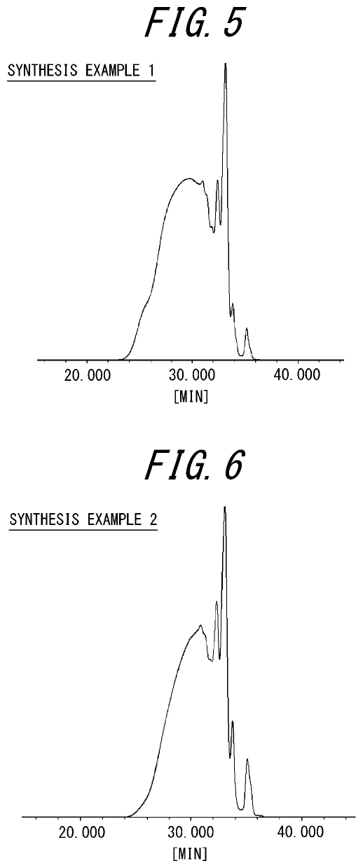

In a 2000 ml four-neck flask equipped with a cooling tube, 164 g (1.52 mol) of m-cresol, 103 g (0.97 mol) of benzaldehyde, 74 g (0.61 mol) of salicylaldehyde, and 8 g of p-toluenesulfonic acid were prepared and dissolved in 300 g of ethanol as a reaction solvent. The resulting solution was then stirred and allowed to react for 16 hours at 80° C. under reflux with a mantle heater. After the reaction, ethyl acetate and water were added, and the solution was washed five times. After the solvent was removed from the remaining resin solution under reduced pressure, vacuum drying was performed to yield 281 g of a novolac phenol resin powder (A1) which was a light red powder. The GPC of the novolac phenol resin (A1) indicated the weight average molecular weight (Mw)=3100. The GPC chart of the novolac phenol resin (A1) is illustrated in FIG. 5.

### Synthesis Example 2: Synthesis of Novolac Phenol Resin (A2)

A novolac phenol resin powder (A2) in the amount of 280 g was obtained by the same method as in Synthesis Example 1 except that the amounts of reaction materials prepared were changed to 164 g (1.52 mol) of m-cresol, 96 g (0.90 mol) of benzaldehyde, and 74 g (0.6 mol) of salicylaldehyde. The GPC of the novolac phenol resin (A2) indicated the weight average molecular weight (Mw)=2250. The GPC chart of the novolac phenol resin (A2) is illustrated in FIG. 6.

### Synthesis Example 3: Synthesis of Novolac Phenol Resin (A3)

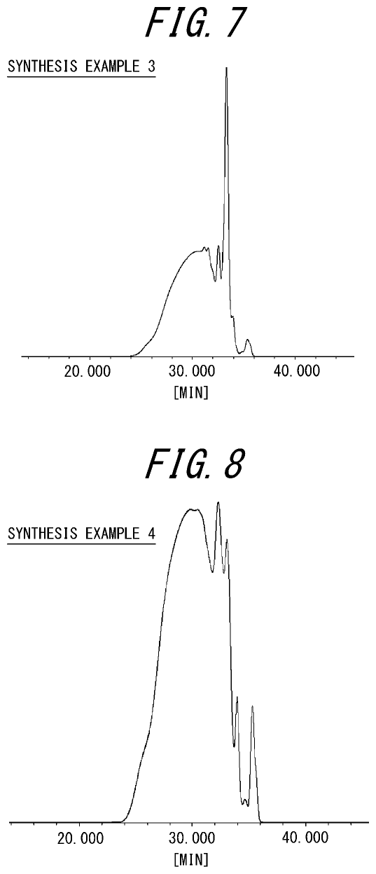

A novolac phenol resin powder (A3) in the amount of 279 g was obtained by the same method as in Synthesis Example 1 except that the amounts of reaction materials prepared were changed to 164 g (1.52 mol) of m-cresol, 117 g (1.10 mol) of benzaldehyde, and 58 g (0.47 mol) of salicylaldehyde. The GPC of the novolac phenol resin (A3) indicated the weight average molecular weight (Mw)=2700. The GPC chart of the novolac phenol resin (A3) is illustrated in FIG. 7.

### Synthesis Example 4: Synthesis of Novolac Phenol Resin (A4)

A novolac phenol resin powder (A4) in the amount of 282 g was obtained by the same method as in Synthesis Example 1 except that the amounts of reaction materials prepared were changed to 164 g (1.52 mol) of m-cresol, 67 g (0.63 mol) of benzaldehyde, and 115 g (0.94 mol) of salicylaldehyde. The GPC of the novolac phenol resin (A4) indicated the weight average molecular weight (Mw)=2900. The GPC chart of the novolac phenol resin (A4) is illustrated in FIG. 8.

### Synthesis Example 5: Synthesis of Novolac Phenol Resin (A5)

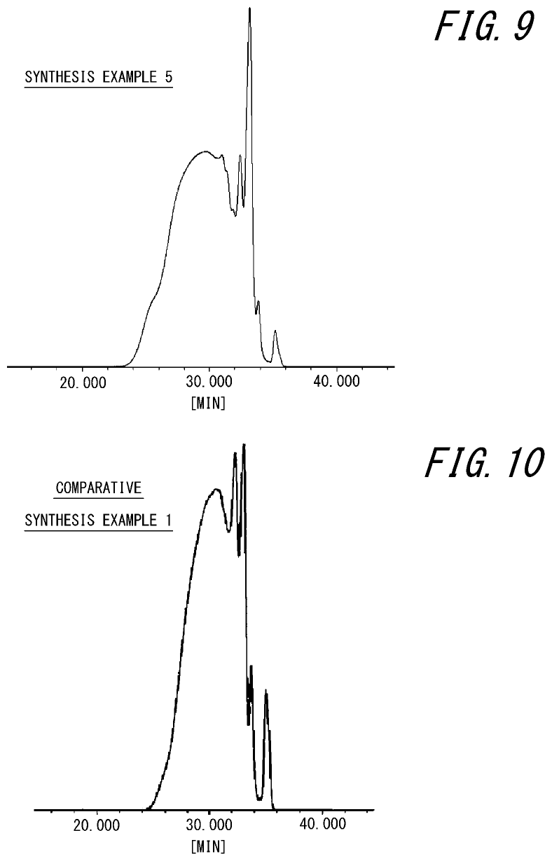

A novolac phenol resin powder (A5) in the amount of 274 g was obtained by the same method as in Synthesis Example 1 except that the reaction solvent was changed to 250 g of ethanol, 30 g of 1-propanol, and 15 g of 2-propanol. The GPC of the novolac phenol resin (A5) indicated the weight average molecular weight (Mw)=3200. The GPC chart of the novolac phenol resin (A5) is illustrated in FIG. 9.

### Comparative Synthesis Example 1: Synthesis of Novolac Phenol Resin (B1)

A novolac phenol resin powder (B1) in the amount of 291 g was obtained by the same method as in Synthesis Example 1 except that the amounts of reaction materials and acid catalyst prepared were changed to 164 g (1.52 mol) of m-cresol, 120 g (1.13 mol) of benzaldehyde, 58 g (0.47 mol) of salicylaldehyde, and 5 g of p-toluenesulfonic acid. The GPC of the novolac phenol resin (B1) indicated the weight average molecular weight (Mw)=3450. The GPC chart of the novolac phenol resin (B1) is illustrated in FIG. 10.

The measurement conditions and evaluation methods are as follows. The verification results of the candidate recipes searched for in Examples 1 to 5 and Comparison 1 are listed in Table 1.

(GPC Measurement Conditions)

Measuring system: “HLC-8220 GPC” manufactured by Tosoh Corporation

Column: “Shodex KF802” (8.0 mm in diameter×300 mm) manufactured by Showa Denko K.K.+“Shodex KF802” (8.0 mm in diameter×300 mm) manufactured by Showa Denko K.K.+“Shodex KF803” (8.0 mm in diameter×300 mm) manufactured by Showa Denko K.K.+“Shodex KF804” (8.0 mm in diameter×300 mm) manufactured by Showa Denko K.K.

Column temperature: 40° C.

Detector: RI (differential refractometer)

Data processing: “GPC-8020 Model II Version 4.30” from Tosoh Corporation

Developing solvent: tetrahydrofuran

Flow rate: 1.0 mL/min

Sample: 0.5% by mass tetrahydrofuran solution in terms of resin solids, filtered through a microfilter

Injection volume: 0.1 mL

Standard sample: monodisperse polystyrenes below

(Standard sample: monodisperse polystyrene)
“A-500” manufactured by Tosoh Corporation
“A-2500” manufactured by Tosoh Corporation
“A-5000” manufactured by Tosoh Corporation
“F-1” manufactured by Tosoh Corporation
“F-2” manufactured by Tosoh Corporation
“F-4” manufactured by Tosoh Corporation
“F-10” manufactured by Tosoh Corporation
“F-20” manufactured by Tosoh Corporation

(Preparation of Test Compositions)

After 4 parts by mass of each of the novolac phenol resins obtained in Synthesis Examples 1 to 5 and Comparative Synthesis Example 1 was dissolved in 6 parts by mass of γ-butyrolactone, the solution was filtered through a 0.5 μm membrane filter to yield a test composition, which was a resin solution.

(Measurement of ADR)

The test compositions were each applied to a silicon wafer with a diameter of 5 inches using a spin coater to a thickness of approximately 1 μm, and then dried at 110° C. for 60 seconds to obtain a wafer with a coating. The resulting wafer was immersed in a developing solution (2.38% tetramethylammonium hydroxide aqueous solution) for 60 seconds and then dried on a hot plate at 110° C. for 60 seconds. The film thickness of each sample was measured before and after immersion in the developing solution, and the difference was divided by 60 to obtain the alkali developability [ADR (A/s)].

(Heat Resistance Evaluation)

The test composition obtained above was applied to a silicon wafer with a diameter of 5 inches using a spin coater to a thickness of approximately 1 μm and dried on a hot plate at 110° C. for 60 seconds. The resin content was scraped off from the resulting wafer and its glass transition temperature (Tg) was measured. The glass transition temperature (Tg) was measured using a differential scanning calorimeter (DSC) (“Q100” available from TA Instruments) under a nitrogen atmosphere, in a temperature range of −100 to 250° C. and a temperature increase of 10° C./min.

(Evaluation Criteria and Evaluation)

The evaluation criteria and evaluation of the novolac phenol resins obtained in Synthesis Examples 1 to 5 and Comparative Synthesis Example 1 were as follows.

Evaluation criteria: Tg as measured by DSC exceeds the target value of 150° C., and the molecular weight and the ADR each fall within the range of −10 to 10% of the target value.

Evaluation: A: the criteria in all of the above three items are satisfied, B: the criterion in one item is not satisfied, C: the criteria in two or more items are not satisfied.

TABLE 1

| | | | | | | | Comparative |
|---|---|---|---|---|---|---|---|
| | | Example 1 | Example 2 | Example 3 | Example 4 | Example 5 | Example 1 |
| Target value | Weight average molecular | 3000 | 2300 | 2800 | 3000 | 3100 | 3000 |
| | weight (Mw) | | | | | | |
| | Tg (° C.) as measured by DSC | ≥150 | ≥150 | ≥150 | ≥150 | ≥150 | ≥150 |
| | ADR(Å/sec) | 1000 | 2300 | 900 | 6000 | 1000 | 1000 |
| Measured value | Weight average molecular | 3100 | 2250 | 2700 | 2900 | 3200 | 3450 |
| | weight (Mw) | | | | | | |
| | Tg (° C.) as measured by DSC | 168 | 154 | 168 | 157 | 170 | 178 |
| | ADR(Å/sec) | 1070 | 2230 | 880 | 5860 | 980 | 580 |

| | | | | | | |
|---|---|---|---|---|---|---|
| Evaluation Result | A | A | A | A | A | C |

As listed in Table 1, all of the evaluation criteria were satisfied in Examples 1 to 5. In other words, when a search process is performed using the present prediction model according to the present example, a novolac phenol resin having a desired property can be searched for more accurately than using the generic prediction model according to the present example.

Although the present disclosure has been described based on the drawings and examples, it should be noted that one skilled in the art can easily make various changes and modifications based on the present disclosure. Therefore, it should be noted that these changes and modifications fall within the scope of the present disclosure. For example, the functions included in each means or each step can be rearranged so as not to be logically inconsistent, and a plurality of means or steps may be combined into one or may be divided.

### REFERENCE SIGNS LIST

10 information processing device
11 control unit
12 storage unit
13 input unit
14 output unit
100 actual data
110 actual data for other applications
120 actual data for predetermined application
210, 220 feature
310 learning process
320 relearning process
400 prediction model

1. A method for searching for a novolac phenol resin that is performed by an information processing device, the method comprising the steps of:
generating a plurality of prediction models corresponding to a plurality of objective variables, using actual data pertaining to a novolac phenol resin; and
searching for a novolac phenol resin having a desired physical property balance by inverse analysis using the prediction models, wherein
the actual data includes a polymer composition, a structural formula, a reaction solvent, and a reaction parameter pertaining to the novolac phenol resin, and
the objective variables include developability, heat resistance, and molecular weight.

2. The method for searching for a novolac phenol resin according to claim 1, wherein in the step of generating the plurality of prediction models, a feature is calculated based on the actual data, and the feature is used as an explanatory variable for the prediction models.

3. The method for searching for a novolac phenol resin according to claim 2, wherein the feature includes at least one of a molecular fingerprint or a descriptor.

4. The method for searching for a novolac phenol resin according to claim 3, wherein the feature further includes information pertaining to an SP value of a solvent.

5. The method for searching for a novolac phenol resin according to claim 1, wherein
the actual data includes actual data of a novolac phenol resin used for a predetermined application and actual data of a novolac phenol resin used for other than the predetermined application, and
in the step of generating the plurality of prediction models,
after the prediction models are generated using the actual data of a novolac phenol resin used for other than the predetermined application, the prediction models are relearned using the actual data of a novolac phenol resin used for the predetermined application.

6. The method for searching for a novolac phenol resin according to claim 5, wherein the predetermined application is semiconductor manufacturing application.

7. An information processing device to search for a novolac phenol resin, the information processing device comprising a control unit, wherein
the control unit
generates a plurality of prediction models corresponding to a plurality of objective variables, using actual data pertaining to a novolac phenol resin, and
searches for a novolac phenol resin having a desired physical property balance by inverse analysis using the prediction models,
the actual data includes a polymer composition, a reaction solvent, and a reaction parameter pertaining to the novolac phenol resin, and
the objective variables include developability, heat resistance, and molecular weight.

8. A non-transitory computer-readable recording medium storing instructions, the instructions, when executed by a processor, causing the processor to:
generate a plurality of prediction models corresponding to a plurality of objective variables, using actual data pertaining to a novolac phenol resin; and
search for a novolac phenol resin having a desired physical property balance by inverse analysis using the prediction models, wherein
the actual data includes a polymer composition, a reaction solvent, and a reaction parameter pertaining to the novolac phenol resin, and
the objective variables include developability, heat resistance, and molecular weight.

9. The method for searching for a novolac phenol resin according to claim 2, wherein
the actual data includes actual data of a novolac phenol resin used for a predetermined application and actual data of a novolac phenol resin used for other than the predetermined application, and
in the step of generating the plurality of prediction models,
after the prediction models are generated using the actual data of a novolac phenol resin used for other than the predetermined application, the prediction models are relearned using the actual data of a novolac phenol resin used for the predetermined application.

10. The method for searching for a novolac phenol resin according to claim 3, wherein
the actual data includes actual data of a novolac phenol resin used for a predetermined application and actual data of a novolac phenol resin used for other than the predetermined application, and
in the step of generating the plurality of prediction models,
after the prediction models are generated using the actual data of a novolac phenol resin used for other than the predetermined application, the prediction models are relearned using the actual data of a novolac phenol resin used for the predetermined application.

11. The method for searching for a novolac phenol resin according to claim 4, wherein
the actual data includes actual data of a novolac phenol resin used for a predetermined application and actual data of a novolac phenol resin used for other than the predetermined application, and
in the step of generating the plurality of prediction models,
after the prediction models are generated using the actual data of a novolac phenol resin used for other than the predetermined application, the prediction models are relearned using the actual data of a novolac phenol resin used for the predetermined application.

12. The method for searching for a novolac phenol resin according to claim 9, wherein the predetermined application is semiconductor manufacturing application.

13. The method for searching for a novolac phenol resin according to claim 10, wherein the predetermined application is semiconductor manufacturing application.

14. The method for searching for a novolac phenol resin according to claim 11, wherein the predetermined application is semiconductor manufacturing application.
